(19) United States
(12) Patent Application Publication
(10) Pub. No.: US 2025/0283853 A1
(43) Pub. Date: Sep. 11, 2025

(54) METHOD AND SYSTEM FOR ANALYZING GAS

(71) Applicant: Technion Research & Development Foundation Limited, Haifa (IL)

(72) Inventors: Israel SCHECHTER, Haifa (IL); Roman SCHUETZ, Haifa (IL)

(73) Assignee: Technion Research & Development Foundation Limited, Haifa (IL)

(21) Appl. No.: 19/073,091

(22) Filed: Mar. 7, 2025

Related U.S. Application Data
(60) Provisional application No. 63/562,274, filed on Mar. 7, 2024.

(51) Int. Cl. G01N 27/64

(52) U.S. Cl. CPC G01N 27/64

(57) ABSTRACT

A system for analyzing a gas, comprises a pair of electrodes arranged to generate an electric field therebetween, a light source system configured to illuminate the gas between the electrodes by a light beam so as to release electrons from the gas by a multiphoton ionization process, and a processor configured to receive from the electrodes an electrical signal generated by electrons accelerating within the electric field, to construct a multiphoton ionization spectrum based on the signal, and to analyze the gas based on the spectrum.

### RELATED APPLICATION(S)

This application claims the benefit of priority of U.S. Provisional Patent Application No. 63/562,274 filed on Mar. 7, 2024, the contents of which are incorporated herein by reference in their entirety.

### FIELD AND BACKGROUND OF THE INVENTION

The present invention, in some embodiments thereof, relates to material analysis and, more particularly, but not exclusively, to a method and system for analyzing gas.

Methods for gas analysis are generally known. Many methods employ absorption spectroscopy, wherein a material to be assayed is exposed to light at a plurality of different wavelengths, and absorption of the radiation is measured to determine its absorption by the material as a function of wavelength. The absorption of the light as a function of the wavelength is known as the “absorption spectrum” of the material.

Also known are methods utilizing a multiphoton ionization effect, wherein two or more photons interacts with a molecule to ionize the molecule. For example, in a two photon multiphoton ionization, a first photon raises an electron in a molecule to an excited state, and a second photon interacts with the molecule before the excited state decays, to add sufficient energy to the electron to free it from the molecule, thereby ionizing the molecule. Multiphoton ionization was applied for the detection of organic compounds in air contained in a chamber held at pressure of 1.3×10⁻⁴ Pa or less, by analyzing signals originating from ions generated by multiphoton ionization of the organic compounds in the chamber [Litani-Barzilai et al., Analytica Chimica Acta 501 (2004) 151-156].

Multiphoton ionization was also applied for the analysis of solids and liquids. U.S. Pat. No. 8,455,813, the contents of which are hereby incorporated by reference, discloses illuminating a solid or liquid sample with pulses of light, generating a multiphoton ionization spectrum for the sample, and processing the spectrum to assay an atom or molecule in the sample.

### SUMMARY OF THE INVENTION

The present embodiments provide a system and a method for analyzing a gas. The technique of the present embodiments can be used in a variety of commercial applications, and can also be used as scientific tool for academic fundamental research in various scientific fields, including, without limitation, studies of the energetic states of substances, the kinetics of chemical reactions, etc. The system of the present embodiments can be used at various ambient conditions including vacuum, and can therefore be employed also in aggressive environments. The technique of the present embodiments can detect spectral patterns without applying analyte preconcentration, chromatographic separation, or specialized environmental controls. This is particularly useful for applications in which it is desired to trace particle monitoring on space missions. Thus, in some embodiments of the present invention the system is installed on a spacecraft.

Thus, according to some embodiments of the invention the present invention there is provided a system for analyzing a gas. The system comprises: a pair of electrodes arranged to generate an electric field therebetween; a light source system configured to illuminate the gas between the electrodes by a light beam so as to release electrons from the gas by a multiphoton ionization process; and a processor configured to receive from the electrodes an electrical signal generated by electrons accelerating within the electric field, to construct a multiphoton ionization spectrum based on the signal, and to analyze the gas based on the spectrum.

According to some embodiments of the invention the system comprises a gas flow chamber, formed with a gas inlet and a gas outlet, wherein the electrodes are positioned in the gas flow chamber.

According to some embodiments of the invention the light source system is configured to vary a wavelength of the light beam.

According to some embodiments of the invention a pressure condition in an interior of the chamber is generally equal to a pressure condition outside the chamber.

According to some embodiments of the invention the system comprises a pump system configured to vary a pressure in the chamber.

According to some embodiments of the invention the system comprises a pump system configured to vary a pressure in the chamber, wherein the processor is configured to receive the electrical signal at different pressure conditions, for each of a plurality of different central wavelengths of the light beam, wherein for each of the central wavelengths, the analysis is based on changes of the signal in response to the pressure variation.

According to some embodiments of the invention the light source system comprises a plurality of light sources.

According to some embodiments of the invention the light source system comprises a plurality of light sources, each configured to generate light at a controllable wavelength.

According to some embodiments of the invention the light source system is configured to illuminate the gas simultaneously by at least two light beams, each generated by a different light source and having a different wavelength.

According to some embodiments of the invention the light source system is configured to generate at least two pulsed light beams, respectively from at least two of the light sources, wherein each pulsed light beam has a different wavelength and wherein pulses of the pulsed light beam are temporally interlaced with each other.

According to some embodiments of the invention the light source system is configured to illuminate the gas during an inflow of through the gas inlet.

According to some embodiments of the invention the gas inlet and gas outlet are opened at all times.

According to some embodiments of the invention the gas flow chamber is telescopic and has a variable length.

According to some embodiments of the invention the electrodes are telescopic and have a variable length.

According to some embodiments of the invention a distance between the electrodes is controllable.

According to some embodiments of the invention a location of the multiphoton ionization process between the electrode is controllable by redirecting the light beam and/or varying a position of the electrodes relative to the light beam.

According to some embodiments of the invention the processor is configured to construct a multiphoton ionization waveform, and to analyze the gas based on both the spectrum and the waveform.

According to some embodiments of the invention the processor is configured to generate ion mobility data based on the waveform, and to analyze the gas based on both the spectrum and the ion mobility data.

According to some embodiments of the invention the processor is synchronized with the light source system and is configured analyze the signal in bins, each bin corresponding to a different central wavelength of the light beam.

According to some embodiments of the invention the processor is configured to apply time-integration separately to each bin, to provide a photocharge associated with the bin.

According to some embodiments of the invention the time-integration is over a period of at least 1 μs, or at least 2 μs, or at least 4 μs, or at least 8 μs, or at least 16 μs, or at least 32 μs, or at least 64 μs, or at least 128 μs, or at least 256 μs, at least 500 μs, or at least 600 μs, or at least 700 μs, or at least 800 μs, or at least 900 μs.

According to some embodiments of the invention the time-integration starts at a time point corresponding to an onset of the illumination.

According to some embodiments of the invention the time-integration starts at a time point corresponding to an onset of illumination at a respective wavelength.

According to some embodiments of the invention the gas flow chamber is a faraday cage chamber.

According to some embodiments of the invention the electric field has intensity of at least 10⁵ V/m.

According to some embodiments of the invention the electric field is constant throughout the wavelength variation.

According to some embodiments of the invention the light beam is generally perpendicularly to the electric field.

According to some embodiments of the invention the system comprises a lens positioned to focus the light beam. According to some embodiments of the invention a focal length of the lens is at least 10% or at least 20% or at least 30% or at least 40% or at least 50% or at least 60% or at least 70% or at least 80% or at least 90% of length of the electrode along a propagation direction of the light beam or larger than the length of the electrode along a propagation direction of the light beam.

According to some embodiments of the invention the light beam has a cross-section of at least 50% or at least 60% or at least 70% or at least 80% or at least 90% of a distance between the electrodes.

According to some embodiments of the invention at least a portion of the wavelength variation is within an ultraviolet range of wavelengths.

According to some embodiments of the invention the wavelength variation is at a sub-nanometer spectral resolution.

According to some embodiments of the invention the light beam is a pulsed light beam.

According to some embodiments of the invention the light source system is configured to generate at least 5 or at least 10 or at least 50 or at least 100 pulses of light at each wavelength.

According to some embodiments of the invention the system comprises a non-differential amplifier for amplifying an electron current collected by the electrodes.

According to some embodiments of the invention the processor is configured to execute a chemometric multivariate analysis model.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 is a schematic illustration of the experimental setup used in experiments performed according to some embodiments of the present invention.

FIG. 2 shows a representative benzene spectrum in nitrogen environment at 150 ppb. The peaks indicated by the dashed arrows were used for calibration in experiments performed according to some embodiments of the present invention: 266.8 nm and 259 nm for high (sub-ppm) and ultralow (sub-ppb) concentrations, respectively.

FIGS. 3A and 3C show calibration curves for sub-ppm range of benzene in air and in nitrogen environments, respectively, as obtained in experiments performed according to some embodiments of the present invention.

FIGS. 3B and 3D show the corresponding spectral region of integration at selected concentrations, as obtained in experiments performed according to some embodiments of the present invention.

FIGS. 4A and 4C show linear calibration curves for sub-ppb range of benzene in air and aniline in nitrogen atmospheres, respectively, as obtained in experiments performed according to some embodiments of the present invention.

FIGS. 4B and 4D show the corresponding spectral region of integration at selected concentrations as obtained in experiments performed according to some embodiments of the present invention.

FIGS. 5A-C are schematic illustrations of a system for analyzing a gas, according to some embodiments of the present invention.

### DESCRIPTION OF SPECIFIC EMBODIMENTS OF THE INVENTION

The present invention, in some embodiments thereof, relates to material analysis and, more particularly, but not exclusively, to a method and system for analyzing gas.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

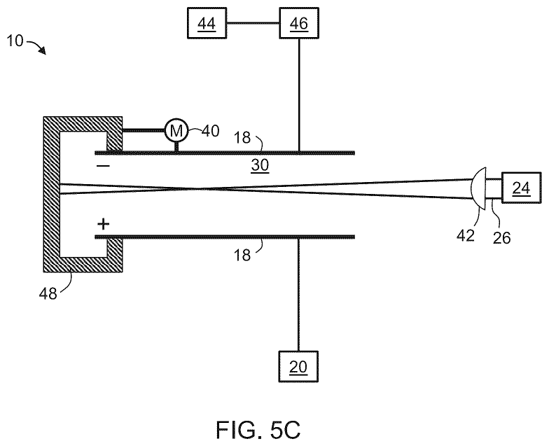

Referring now to the drawings, FIGS. 5A-C are schematic illustrations of a system 10 for analyzing a gas 30, according to some embodiments of the present invention. System 10 optionally and preferably comprises a gas flow chamber 12, formed with a gas inlet 14 and a gas outlet 16. While the embodiments below are described with a particular emphasis to a configuration in which system 10 comprises gas flow chamber 12, it is to be understood that system 10 can operate in the absence of gas flow chamber, as illustrated in FIG. 5C and further detailed hereinbelow.

The size of gas flow chamber 12 is typically from about 5 cm to about 50 cm, in length, from about 0.5 cm to about 10 cm in height, and from about 0.5 cm, to about 20 cm in width. Other sizes for gas flow chamber 12 are also contemplated in some embodiments of the present invention. In some embodiments of the present invention gas flow chamber 12 is telescopic so that its length can be varied, as illustrated in FIG. 5B. These embodiments are useful when system 10 is hand held or otherwise temporarily deployable in an indoor or outdoor location of interest.

The conditions in chamber 12 need not be pressurized. For example, the pressure condition in the interior of chamber 12 can be generally the same as the pressure condition outside chamber 12. Alternatively, system 10 can comprise a pump 34 configured to vary (increase or decrease) the pressure in the interior of chamber 12.

The gas inlet 14 and gas outlet 16 are optionally and preferably formed at opposite walls of gas flow chamber 12, as illustrated in FIGS. 5A-B. Alternatively, they can be formed on the same wall or on adjacent walls thereof. Typically, but not necessarily, the gas inlet 14 and outlet 16 are constituted to allow inflow and outflow of gas 30, respectively, at flowrates ranging from a fraction (e.g., 0.5) of a liter per minute to a few tens (e.g., 30, 40, 50, etc.) of a liters per minute. Other flowrates are also contemplated. Gas inlet 14 and gas outlet 16 can be set to be opened at all times. Alternatively, system 10 can comprise controllable valves 36 and 38 for opening and closing or otherwise controlling the inflow and outflow of gas 30 into and out of chamber 12.

In some embodiments of the present invention gas flow chamber 12 functions as a Faraday cage. This can be achieved by constructing the walls of chamber 12 using a conductive material, so as to effectively shield the interior of chamber 12 from electromagnetic fields that may exist outside chamber 12.

System 10 further comprises a pair of electrodes 18 arranged opposite to each other so as to generate an electric field between electrodes 18 once applied by voltage from a power source 20. In embodiments in which gas flow chamber 12 is employed, electrodes 18 are positioned inside gas flow chamber 12. In embodiments in which gas flow chamber 12 is not employed, electrodes 18 can be deployed openly within an environment containing gas 30. For example, electrodes 18 can be mounted on a non-conductive structure 48 (see FIG. 5C) maintaining a separation between them.

The optional Faraday cage functionality of chamber 12 is advantageous since it reduces or prevents interference of the generated the electric field with other electric fields.

The present embodiments contemplate various ranges for the intensity of the generated electric field. In some embodiments of the present invention the intensity is at least 10⁵ V/m or at least 10⁶ V/m or higher. In other embodiments, the intensity is lower than 9·10⁴ V/m or lower than 10⁴ V/m or lower than 10³ V/m or lower than 10² V/m or even lower than 10 V/m. Embodiments in which the intensity of the generated electric field is low are useful when electrodes 18 are placed in chamber 12 and when the pressure within chamber 12 is substantially lower (e.g., at least 2, 3, 4, 5, or 10 times lower) than the pressure outside chamber 12.

The electric field intensity is optionally and preferably of constant intensity, but temporal variations of the electric field intensity are also contemplated. In the schematic illustration in FIGS. 5A-C, the voltage is applied such that the electrical potential of the lower electrode is higher than the electrical potential of the upper electrode, but opposite electrical configuration is also contemplated.

Typically, electrodes 18 are connectable to power source 20 by means of a connector 22 formed in the wall of chamber 12. In some embodiments of the present invention power source 20 is part of system 10, and in some embodiments of the present invention system 10 is provided without a power source and is connected to power source 20 before operation. Typical dimensions of electrodes 18 are, without limitation, from about 1 cm to about 30 cm or from about 10 cm to about 30 cm in length, and from about 1 mm to about 5 cm or from about 1 cm to about 5 cm in width. Other dimensions for electrodes 18 are also contemplated in some embodiments of the present invention. The separation between the electrodes, as measured perpendicularly to the surface of electrodes 18 is from a fraction (e.g., 0.5) of a centimeter to about 5 cm. Other values for the distance between electrodes 18 are also contemplated in some embodiments of the present invention. In embodiments in which chamber 12 is telescopic (FIG. 5B), electrodes 18 are optionally and preferably also telescopic with a variable length. In some embodiments of the present invention system 10 comprises a stage 40, such as, but not limited to, a stage controllable by a motor, configured for varying the distance between electrodes 18 and/or the length of electrodes 18.

System 10 also comprises a light source system 24 configured to illuminate gas 30 between electrodes 18 by a light beam 26 so as to release electrons from the gas 30 by a multiphoton ionization process. In embodiments in which gas flow chamber 12 is employed, the gas is optionally and preferably illuminated by light beam 26 during the inflow of gas though inlet 14. Alternatively, the inlet and/or outlet can be closed (e.g., by valves 36 and 38) before illuminating the gas. In embodiments in which gas flow chamber 12 is not employed, the gas is optionally and preferably illuminated by light beam 26 while electrodes 18 are deployed in an environment and are exposed to gas 30. Preferably, light beam 26 is generally (e.g., within tolerance of ±10%) perpendicularly to the electric field between electrodes 18.

In some embodiments of the present invention light beam 26 has a varying wavelength The wavelength variation preferably spans over a wavelength range of at least 50 nm or at least 75 nm or at least 100 nm or at least 125 nm or at least 150 nm or at least 200 nm or more. At least a portion of the wavelength variation is optionally and preferably within the ultraviolet range of wavelengths. Preferably, the wavelength variation is at a sub-nanometer spectral resolution.

In embodiments in which gas flow chamber 12 is employed, light source system 24 is typically positioned outside chamber 12 in which case the wall receiving light beam 26 is made of a material that transparent to wavelengths that induce multiphoton ionization in gas 30. Alternatively, a window 28 can be formed on the receiving wall, which window can be covered by a material that transparent to such wavelengths. Representative examples of such a transparent material include, without limitation, quartz, fused silica, calcium fluoride, magnesium fluoride and the like. A typical thickness for window 28 is from about 1 μm to about 3 cm, or from about 1 μm to about 1 cm, or from about 1 μm to about 5 mm. Typical lateral dimensions of window 28 are from a fraction (e.g., 0.5) of a centimeter to several centimeters in length and from a fraction (e.g., 0.5) of a centimeter to several centimeters in width. Optionally, but not necessarily, the wall of chamber 10 that is opposite to the receiving wall is also made transparent, or formed with another window 32 that can be similar in material and dimensions to window 28.

The wavelengths and intensity of the light generated by light source system 24 are selected to induce multiphoton ionization in gas 30. For example, the wavelengths can be in the ultraviolet range (e.g., from about 100 nm to about 400), or the visible range (e.g., from about 400 nm to about 750 nm), and may also be in the infrared range (from about 750 to about 1500 nm). Typically, light source system 24 is a laser system, providing optical intensity sufficient to induce multiphoton ionization, e.g., from a few tenths of kW to a few MW per square millimeter.

Light source system 24 can be embodied in more than one way. In some embodiments of the present invention light source system 24 comprises a single light source, which is controllable in a manner that the wavelength and optionally and preferably at least one of the intensity and illumination mode (e.g., continuous, pulsed) can be varied by transmitting to system 24 an appropriate control signal. These embodiments are useful from the standpoint of simplicity. In other embodiments, light source system 24 comprises a plurality of light sources. One or more of these light sources can be configured to generate light at a fixed wavelength or, alternatively, and more preferably, one or more of these light sources can be controllable in a manner that the wavelength and optionally and preferably at least one of the intensity and illumination mode (e.g., continuous, pulsed) can be varied by transmitting an appropriate control signal. In some embodiments of the present invention all light sources generate light at the same wavelength, and in some embodiments of the present invention at least two light sources generate light at different wavelengths. The advantage of these embodiments is that they allow illuminating gas 30 simultaneously by two or more light beams, each generated by a different light source and having a different wavelength.

Preferably, light beam 26 is a pulsed light beam. Typical repetition rates, durations, and pulse energies of the generated pulses are: from about 1 Hz to a few tens of Hz, from a few ps to a few ns per pulse, and from a few μJ to a few mJ per pulse, respectively. Other repetition rates, durations, and pulse energies are also contemplated. Light source system 24 preferably generates 5 or more pulses of light at each wavelength. In embodiments in which light source system 24 comprises a plurality of light sources, the pulses generated by different light sources can be temporally interlaced with each other. For example, when two light sources are employed, a train of pulses p1, p2, p3, etc. can be applied, where odd-numbered pulses are generated by one light source and even-numbered pulses are generated by another light source.

In some embodiments of the present invention system 10 comprises a focusing optical element 42, such as, but not limited to, a lens or a grating, positioned to focus light beam 26 onto a region within the inter-electrode space. With respect to the inter-electrode distance, the region is preferably centered, so that its center is at generally equal distances with both electrodes. Preferably, the focusing is mild, with a relatively long length. This is advantageous since it reduces the likelihood plasma creation in the gas phase between the electrodes, while maintaining sufficiently high electromagnetic field densities along the beam. In some embodiments of the present invention the focal length of focusing optical element 42 is at least 10% or at least 20% or at least 30% or at least 40% or at least 50% or at least 60% or at least 70% or at least 80% or at least 90% of the length of the electrode along a propagation direction of the light beam or larger than the length of electrode 18 along the propagation direction of light beam 26. Light source system 24 and focusing optical element 42 are optionally configured to ensure that the transverse cross-section of beam 26 is at least 50% or at least 60% or at least 70% or at least 80% or at least 90% of the distance between electrodes 18.

The present embodiments also contemplate configuration whereby the location of the multiphoton ionization process between electrode 18 is controllable. This can be achieved by providing system 10 with a functionality that allows adjusting at least one the inter-electrode distance, the position of element 42, and the focal length of element 42 so as to select a location between the electrodes onto which the beam 26 is focused. The position of element 42 can be adjusted by means of a stage or the like (not shown). The focal length of element 42 can be varied by means of known techniques, including, without limitation, use of liquid lenses, deformable mirrors, electrowetting lenses, adaptive optics, zoom lenses, and the like.

System 10 optionally and preferably also comprises a processor 44 having a circuit configured to receive from electrodes 18 an electrical signal generated by electrons accelerating within the electric field, to construct a multiphoton ionization spectrum based on the signal, and to analyze the gas based on the spectrum. The analysis optionally and preferably includes determining at least one or a plurality of gas components in gas 30. For example, processor 44 can execute a chemometric multivariate analysis model.

Thus, unlike traditional techniques in which the produced ions are analyzed by mass spectrometers, the present embodiments analyze electrical current that is generated by the electrons. In some embodiments of the present invention the electron current collected by electrodes 18 is amplified by an amplifier 46, such as, but not limited to, a non-differential amplifier.

In embodiments in which the pressure within chamber 12 is controlled (e.g., by means of pump 34), processor 44 optionally and preferably receives electrical signal at different pressure conditions, for each of a plurality of different central wavelengths of light beam 26, wherein for each of the central wavelengths, the analysis is based on changes of the signal in response to the pressure variation. Processor 44 can construct a multiphoton ionization waveform. Based on the spectrum and the waveform, processor 44 can analyze gas 30. Processor 44 can also generate, based on the waveform, ion mobility data. Based on the spectrum and ion mobility data, processor 44 can analyze gas 30.

Processor 44 is optionally and preferably synchronized with light source system 24. In this case, processor 44 can analyze the signal in bins, wherein each bin corresponds to a different central wavelength of light beam. Preferably, processor 44 applies time-integration separately to each bin, to provide a photocharge associated with that bin. The time-integration is optionally and preferably over a period of at least 1 μs, or at least 2 μs, or at least 4 μs, or at least 8 μs, or at least 16 μs, or at least 32 μs, or at least 64 μs, or at least 128 μs, or at least 256 μs, at least 500 μs, or at least 600 μs, or at least 700 μs, or at least 800 μs, or at least 900 μs. The time-integration can begin at a time point corresponding to the onset of the illumination at the respective wavelength.

The system of the present invention has a wide array of potential applications that can significantly benefit various fields, for example, in health, environmental monitoring, food quality control, product authentication and the like. The system of the present embodiments can be used for environmental monitoring, e.g., by providing real-time (e.g., within minutes of seconds) detection of trace pollutants and toxic substances, thereby aiding in regulatory compliance and atmospheric studies. The system of the present embodiments can also be used in industrial process control, by monitoring VOCs and trace gases in various industrial settings, thus ensuring safety and efficiency. The system of the present embodiments can also be used in the field of homeland security and defense, for example, as a selective tool for detecting explosives and chemical agents, thereby enhancing security measures. The system of the present embodiments can also be used for medical diagnostics, for example, in non-invasive breath analysis for disease biomarkers, thereby contributing to early diagnosis and treatment. The system of the present embodiments can also be used in the food and beverage industry for quality control, in the automotive industry for emissions testing, and in consumer products for safety monitoring.

One of the most promising applications lies in the early detection of diseases through the analysis of multiphoton ionization spectrum of exhaled breath. By capturing and analyzing the unique spectral signatures associated with specific biomarkers in exhalation, healthcare professionals could identify the presence of certain diseases at a much earlier stage than conventional diagnostic methods allow. This early warning capability can facilitate timely interventions and personalized treatment plans, ultimately improving patient outcomes.

In the realm of environmental safety, the system of the present embodiments can serve as a useful tool for the early warning of leaks involving volatile and hazardous materials, such as Benzene, Toluene, Ethylbenzene, and Xylenes (BTEX) compounds. By monitoring the air quality for specific spectral signatures indicative of these harmful substances, the system of the present embodiments can provide alerts, optionally and preferably real-time alerts, allowing swift responses to prevent environmental contamination and protect public health.

Another potential application of the system of the present embodiments relates to the detection of very low concentrations of nitrogen oxides (NOx) in the atmosphere. Capable of operating in the parts per billion (ppb) and even parts per trillion (ppt) range some embodiments, the system can help regulatory bodies and environmental agencies monitor air quality more effectively, ensuring compliance with environmental standards and safeguarding community health.

In the food industry, the ability to obtain an aroma and odor material fingerprint through the analysis of the multiphoton ionization spectrum offers valuable insights for quality control and spoilage detection. By characterizing volatile compounds emitted by food materials, producers can monitor for signs of spoilage or degradation, ensuring that consumers receive high-quality products. This application not only enhances food safety but also helps maintain brand integrity.

Another potential application of the system of the present embodiments relates to the authentication of aromatic products, including, without limitation, perfumes, wines, alcoholic beverages, and tobacco products. By analyzing the multiphoton ionization spectrum of these items, fingerprints can be identified, and can be used to authenticate the items and/or detect counterfeits, and/or ensure that the products meet quality standards. This capability not only protects consumers but also upholds the reputation of brands in highly competitive markets.

The system of the present embodiments can also be used for detecting allergenic compounds within food products. For example, the presence of peanuts or other allergens in various food items can be identified through the analysis of the multiphoton ionization spectrum of emitted vapors. This application is particularly useful for the safety of individuals with food allergies, as it can facilitate better labeling practices and improve overall food safety measures.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Sub-PPB Detection with Gas-Phase Multiphoton Electron Extraction Spectroscopy Under Ambient Conditions

This Example introduces application of MEES in the gas phase (Gas-phase MEES), which in some embodiments of the present invention can be used for quantitative detection of gas traces at sub-part per billion (sub-PPB) concentrations under ambient atmospheric conditions. The experimental setup described below utilizes resonant multiphoton ionization processes using ns laser pulses under high electrical field. The generated photoelectron charges are recorded as a function of the laser's wavelength. The experimental data presented herein show improved sensitivity of Gas-phase MEES, achieving spectral resolution with resonant peak widths less than 0.02 nm FWHM. This Example presents results from quantitative analysis of benzene and aniline, two industrially and environmentally significant compounds, demonstrating linear responses in the sub-PPM and sub-PPB ranges. The enhanced sensitivity and resolution of Gas-phase MEES can be used in many scientific and industrial fields, including, without limitation, environmental monitoring, industrial safety, security screening, and medical diagnostics. This Example demonstrates the advantage of Gas-phase MEES over many traditional optical spectroscopic methods and its potential in direct gas-trace sensing in ambient atmosphere.

### Introduction

The precise detection, measurement, and monitoring of trace concentrations of substances in gaseous environments are useful for a multitude of applications including, without limitation, applications that impact public safety, environmental health, and industrial processes. Example applications include, but are not limited to, enhancing security, identification of explosive materials, and tracking atmospheric pollutants. Additional uses of trace gas detection include detecting hazardous gas leaks, e.g., to ensure industrial safety, and medical diagnostics, e.g., breath analysis.

Recent advances in breath analysis have shown promising results for non-invasive disease diagnosis and monitoring, leveraging the detection of volatile organic compounds (VOCs) as biomarkers for various conditions, including respiratory diseases, cancer, and metabolic disorders [1-4]. This burgeoning field, often referred to as “breathomics,” employs conventional techniques such as gas chromatography-mass spectrometry (GC-MS) and electronic noses (e-noses) [5,6]. It is recognized that breath analysis faces significant challenges, such as the complexity of breath composition, the influence of external factors on VOC concentrations, and the need for standardization in sampling and analysis [7]. Moreover, disease-specific biomarkers are typically present at the low concentrations making it difficult to distinguish them in-situ under ambient conditions from the myriad of other compounds present in breath [8].

The inventers realized that contemporary direct and on-line analytical techniques fall short when it comes to accurately detecting and quantifying trace concentrations in the sub-parts per billion (sub-ppb) range under ambient conditions, in particular when both high selectivity and sensitivity are required. Current technologies thus employ either preconcentration of analytes or establishment of controlled environments, which may involve high vacuum conditions and regulated temperatures, to achieve the desired measurement precision [10-12]. While, some laboratory analytical instruments allow for extremely low limits of detection for some contaminants in gas samples [13], a preconcentration step is oftentimes required to achieve a sufficient sensitivity. The inventors realized that a preconcentration step compromises the response time and is only effective for some specific chemical groups.

Although fast chromatographic instruments are available, good separation of mixtures requires considerable chromatographic retention times. The regular optical spectroscopy technologies, including laser-based systems, provide real on-line detection. However, the inventors found that their specificity and their ability to handle mixtures are moderate, and that their limits of detection are above the ppb range. The sensitivity of laser induced fluorescence-based detectors is lower, but is useful only for fluorescing compounds. The inventors have therefore searched for a detection technology, which has the potential of sensitive on-line measurements, and which provides rich analytical information that can result is good identifiability capability and handling of mixtures.

Multiphoton ionization (MPI) is a known process in which several photons from a short laser pulse are simultaneously absorbed by a molecule, resulting in ionization. The process has mainly been used in mass spectrometry (MS), where molecular masses and their fragments have been identified [14-16]. In most MPI-MS configurations, a single laser wavelength is utilized, however, resonance enhanced ionization and two laser ionization have also been reported. The inventors found that these setups are limited in terms of the number of analyte molecules, because ionization takes place in a single small spot, due to the need to manipulate the generated ions in mass spectrometric detectors. The inventors found that the above mass spectrometric methods are also limited in that they require high vacuum conditions, so as to provide long mean free pass of the ions. Known is a method in which time-dependent mirror charges are monitored following illumination by an unfocused laser beam [17]. In this method, the laser beam generates ions along its pass, and they move towards a nearby negative electrode. The distance to the electrode is about 1 cm, and only a moderate vacuum, about 10⁻⁵ torr, is employed. This time-of-flight is too short to allow effective analysis, hence the measurement of induced mirror charges. Thus in this method instead of measuring a single value (time-of-flight, arrival location, etc.) for the identification, numerous data were collected for each ion during its flight, partially compensating for the errors due to the short time-of-flight. While this method allows on-line operation and ionization of a large number of molecules, the inventors found that it severely compromises the mass identification, wherein only simple mixtures can be properly resolved.

Multiphoton ionization under ambient conditions (atmospheric pressure, temperature, and composition), performed at a single laser wavelength, has also been applied to analysis of solid and liquid surfaces [18-21]. The results indicated high sensitivity to a variety of molecules, but no identification was possible under such conditions.

While conceiving the present invention the inventors realized that Multiphoton Electron Extraction Spectroscopy (MEES) can be used to overcome at least some of the aforementioned challenges. MEES is an analytical method that was suggested for direct analysis of solids and liquids under ambient conditions. MEES utilizes short ultraviolet (UV) laser pulses to excite and ionize samples in a multiphoton process. Unlike in MS methods, photoelectrons are recorded, which is much simpler and lower cost as compared to ions. The resulted time-resolved current is integrated, providing the total emitted photocharge. Detection is based on the spectrum of photocharges as a function of the laser wavelength. In this kind of spectroscopy, numerous sharp peaks are observed. They are related to resonant transitions between the molecular electronic and vibronic energy levels of the analyte. A peak is generated when the energy of any combination of photons is equal to the transition of the molecule to any of its excited states. Therefore, the number of peaks in this spectroscopy is much larger than in absorption or emission spectroscopies. Very high energy levels of the molecule can also be revealed. The density of peaks (number of peaks per a given wavelength range) in MEES is much larger than in all single photon spectroscopies. This results in more analytical information, which is beneficial for identification and for handling of mixtures.

In MEES, the normal composition of ambient air does not interfere with many organic and inorganic analytes, simply because the ionization potential the formers is higher. It means that more photons are needed for their ionization, and their multiphoton ionization cross section is much lower. Consequently, their ionization probability is much lower [16].

Heretofore, MEES has only been developed and characterized for analysis of surfaces [22-26], reporting fmole sensitivity for direct analysis under ambient conditions. The high density of sharp peaks allows compound fingerprinting and therefor improves the capability to identify compounds [27]. The analytical performance of MEES was compared to that of most common optical spectroscopies, and was found to be superior in terms of both sensitivity and analytical information [27].

Following is a description of an experimental setup for the application of MEES to the gas phase. The results section below demonstrates signal linearity over a wide concentration range for high pressure compounds, and sensitivity in the sub ppb range for low pressure compounds.

### Materials and Methods

### Experimental Setup

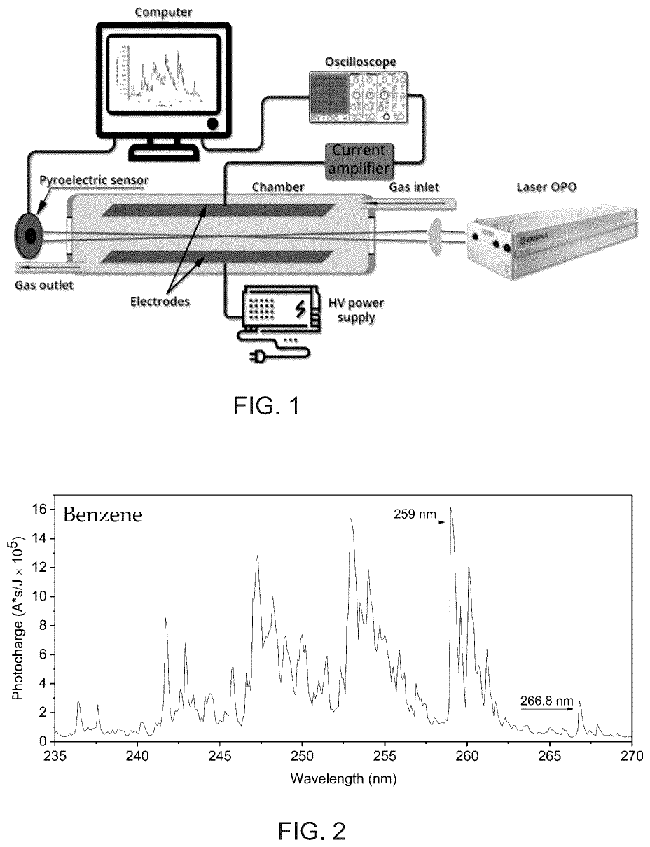

A Gas-phase Multiphoton Electron Extraction Spectroscopy (Gas-MEES) system was assembled according to some embodiments of the present invention to facilitate the measurement of molecular traces in gases at ambient conditions. FIG. 1 presents a block diagram of the experimental setup. In a faraday cage flow chamber, two electrodes 22 cm long and 2.5 cm wide were positioned 1-2 cm apart (the distance is variable). A high voltage supply (PS325, Stanford Research Systems) was connected to one of the electrodes and can provide up to 5 kV/cm electric field. The second electrode was connected to a current amplifier (Variable Gain Low Noise Current Amplifier DLPCA-200, FEMTO, Messtechnik GmbH) and an oscilloscope (Picoscope 3000 series, Pico Technology) as the time-resolved detecting system. Nitrogen gas of high-grade purity (99.999%) was used as a carrier gas and air was provided by the institute's compressor to the lab.

The light source was a solid-state OPO pulsed laser (NT 342/3/UVE EKSPLA, Vilnius, Lithuania). The laser offers a wide range of wavelengths in the UV from 192-410 nm with 0.02 nm step resolution and a repetition rate of 10 Hz, whereas the pulse duration is about 2 ns. The laser pulse energy is between 0.2 mJ to 2.5 mJ. The intensity of the laser at each wavelength was monitored by a pyroelectric sensor (Ophir Laserstar PE-10-sh-v2, Israel). The beam was introduced to the chamber through a 1-inch quartz window. The configuration included two electrodes with a 2 kV/cm electrical field applied between them. The laser beam was mildly focused by a lens (f=30 cm) in the center between the electrodes. The mild focusing by such long focal length was selected in order to avoid plasma creation in the gas phase between the electrodes, while still achieving high EM field densities along the beam.

### Gas Flow Chamber System

A flow design of the chamber was chosen due to several advantages over static design. First, it simulates an on-line sensor setup. The flow design also ensures uniform exposure of the gas to the laser pulses, mitigating the effects of diffusion, adsorption/desorption processes, and reducing the influence of potential photochemical reactions. This setup significantly reduced the risk of contamination, providing a controlled and consistent experimental environment.

### Representing Analytes

Benzene and aniline were chosen as representing analytes due to their significance in organic chemistry, industrial relevance, and environmental impact. Benzene, a typical aromatic hydrocarbon, was used to assess the system's performance in the sub-ppm and sub-ppb concentration ranges in both pure nitrogen and air. Additionally, aniline, an aromatic amine, also significant due to a wide application range such as pharmaceuticals, dyes, and organic synthesis, was employed for studying the sub-ppb range. The accurate detection and quantification of these VOCs are critical for health and safety, compliance with regulatory standards, and environmental monitoring.

### Measurement and Data Acquisition

Upon photo-ionization, the generated molecular ions and photoelectrons were collected by the electrodes. The resultant time-dependent current was then amplified using a current amplifier and recorded with a pico-scope. The pico-scope was synchronized with the laser pulses by adjusting its trigger.

The setup operation, data acquisition and processing were automated by software complex developed using VB.NET code. After recording the waveforms of the photocurrent as a function of time, these data were integrated to obtain the total photo charge as a function of laser wavelength. Laser intensity (from the power-meter) was also recorded, so that normalization of the total photo charge to the beam energy could be carried out.

### Sample Preparation and Dilution

Two distinct methods were employed for the preparation and dilution of the specimen gases to achieve the desired concentration ranges:

Permeation Tube Method: PTFE permeation tubes were used in experiments with benzene. The permeation tube was placed in a glass bubbler before the chamber inlet. A stable flow (nanograms-per-minute) of analyte vapor was emitted through the tube wall at constant temperature [28]. The emission rate of the tube was determined by measuring the rate of weight loss [28] during a long-term period. The temperature of the permeation tube was kept constant to maintain a constant output, while the carrier gas flow rate was adjusted to achieve the required concentrations. In order to cover both concentration ranges, sub-ppm and sup-ppb, yet keeping the carrier gas flows economically moderate (nitrogen up to 3 L/min and air up to 26 L/min), different sizes of permeation tubes were used, big and small, respectively.

Syringe Pump Method ([29]): This method was utilized for aniline at sub-ppb concentration ranges, characterized by its low efficiency to penetrate the permeation tubes. The syringe pump (Standard Infuse/Withdraw Pump 11 Elite Programmable Syringe Pump Harvard Apparatus, Holliston, Massachusetts, USA) with a micro syringe (2 μL Syringe Model 7102, Knurled Hub, Switzerland) delivered different precise dilutions of aniline in ethanol, with the gas flow rate held constant to attain the ultra-low concentrations needed for our measurements.

### Results

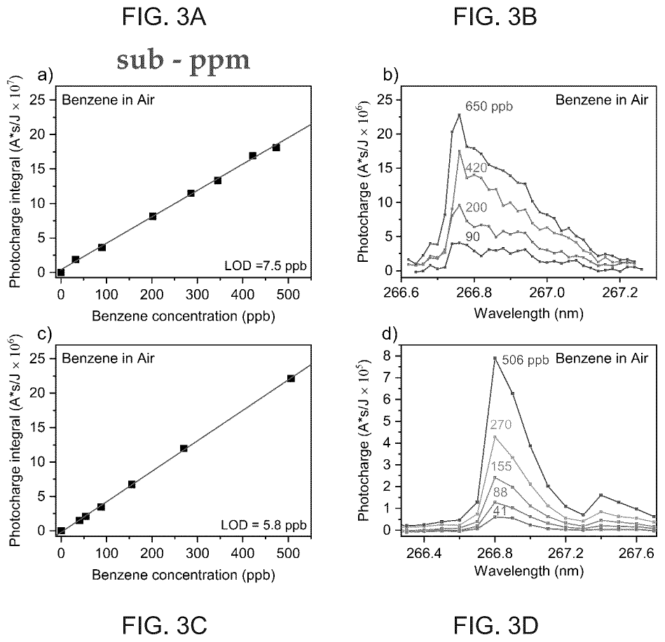

The Gas-MEES setup of the present embodiments was used to measure molecular traces in gases under atmospheric conditions with sub-ppb sensitivity. A flow chamber ensured uniform gas exposure and minimize contamination, and a pulsed laser was used for the ionization of gas molecules between two electrodes (FIG. 1). The resultant current waveforms were recorded over time, and the integrals of these peaks were plotted to generate a photocharge spectrum as a function of the laser wavelength (FIG. 2). In the presence of atmospheric oxygen, the photoelectron pulse is considerably lower compared to that in nitrogen environment. However, with the increase of the voltage between the electrodes, the photoelectron pulse current rises up. Increased electrode voltages can lead to saturations of the detection and to nonlinear calibration characteristics of the concentrations. The instrumental parameters were therefore adjusted for the specific range of sensitivity. In this Example, the integral of the sharp electron peak was obtained.

FIG. 2 presents MEES spectrum of Benzene measured at 0.1 nm resolution. This low resolution was used in order to present the full spectrum in a wide range. However, the laser's best resolution is 0.02 nm, which was actually used in the quantitative measurements. The high resolution spectra allow for much better analyte identification. For quantification purposes, spectral peaks that were neither too intense (to avoid system saturation) nor overlapped significantly with the spectral features from the carrier gas were selected. Accordingly, benzene calibration was done on the peaks at 266.80 nm and 259.00 nm for high (sub-ppm) and ultralow (sub-ppb) concentrations, respectively.

In parallel, aniline calibration was based on the integrated signals in the range of 285.82 nm and used for the ultrahigh sub-ppb region.

### Sub-PPM Range

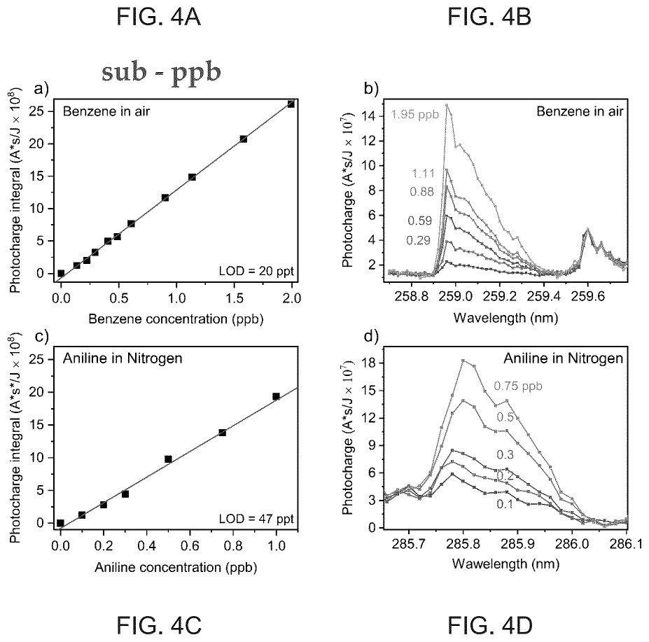

Initially measurements were performed to demonstrate sub-ppm range using benzene, a compound of high vapor pressure, so as to validate the linear response of the Gas-MEES system in ambient air and in nitrogen. Utilizing a permeation tube, the inventors were able to regulate benzene concentrations within the 0-650 ppb range by varying the flow rate of the carrier gas. The results are shown in FIGS. 3A-D. As shown, there is a linear relationship between the photocharges and benzene concentration, demonstrating the capability of the technique of the present embodiments to quantitatively detect in this region in air and in nitrogen. The currently determined 95% based limits of detection (LODs) were 7.5 ppb and 5.8 ppb, respectively.

### Sub-PPB Range

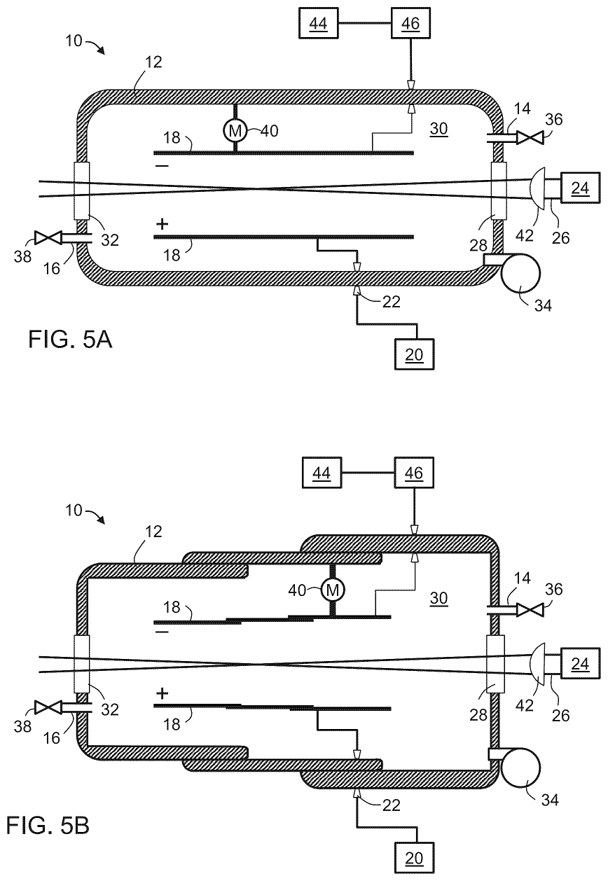

In order to explore the sub-ppb sensitivity range, aniline was analyzed. Aniline is a compound with a relatively low vapor pressure. Unlike benzene, aniline showed no mass loss through the walls of the permeation tubes and subsequently no spectrum could be obtained using this approach. Therefore, the syringe pump method was applied for aniline. By employing a syringe pump and varying dilutions in ethanol, the aniline concentrations was controlled below 1 ppb. The resulted calibration curves for benzene and aniline in air and in nitrogen environments are shown in FIGS. 4A and 4C. The results exhibit a linear response, extending the system's demonstrated sensitivity to the sub-ppb level. The 95% based LOD's for the presented calibration curves of Benzene in air and Aniline in nitrogen are 20 ppt and 47 ppt, respectively.

The corresponding Gas-MEES spectra are also shown in FIGS. 4B and 4C, demonstrating rich MEES spectral features, which, together with the high spectral resolution, can be used for material fingerprinting and identification of the analyte.

The capability to quantify molecular traces at sub-ppb concentrations under ambient conditions in air or nitrogen atmosphere, without the need for preconcentration or controlled environments, has been demonstrated. While lower concentrations have not been investigated, due to lack of gas samples at such concentrations, it is envisioned that the system of the present embodiments can provide sensitivity of ppt concentrations or less.

### Conclusions

This Example demonstrated a development and implementation of Gas-phase Multiphoton Electron Extraction Spectroscopy system according to some embodiments of the present invention. This Example also demonstrated the utility of the system of the present embodiments for the quantitative detection of volatile and non-volatile organic compounds in ambient air at both sub-ppm and sub-ppb concentration.

The results indicate that Gas-phase MEES surpasses the detection capabilities of many existing on-line analytical spectroscopic methods in terms of sensitivity and spectral information. The ability to detect benzene and aniline with such precision, without the necessity of analyte preconcentration, chromatographic separation, or specialized environmental controls, implies on the potential of this technology in trace gas monitoring.

The gas-MEES of the present embodiments can be used in applications across various sectors. These include environmental protection, where precise monitoring of air pollutants is useful; industrial safety, for the detection of gas leaks and ensuring compliance with health regulations and security, for identifying explosive or harmful chemical agents. Additionally, the gas-MEES of the present embodiments can be used in the medical field, particularly, but not exclusively, in the development of breath analysis techniques for disease diagnosis and monitoring.

It is to be understood that aside for the compounds detected in this Example, the system of the present embodiments can be used for the detection and optionally and preferably identification of many other compounds. Due to the abundancy of sharp peaks, the system of the present embodiments can also be used for the detection and optionally and preferably identification of analyte mixtures. In some embodiments of the present invention libraries of MEES spectra of a variety of compounds are utilized for the identification of compounds. The system of the present embodiments can be provided as a laboratory system or as a portable system for in-field measurements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

[1] Das, S.; Pal, M. Review—Non-Invasive Monitoring of Human Health by Exhaled Breath Analysis: A Comprehensive Review. J Electrochem Soc 2020, 167, 037562, doi:10.1149/1945-7111/AB67A6.
[2] Li, Y.; Wei, X.; Zhou, Y.; Wang, J.; You, R. Research Progress of Electronic Nose Technology in Exhaled Breath Disease Analysis. Microsyst Nanoeng 2023, 9, doi:10.1038/S41378-023-00594-0.
[3] Pauling, L.; Robinson, A. B.; Teranishi, R.; Cary, P. Quantitative Analysis of Urine Vapor and Breath by Gas-Liquid Partition Chromatography. Proc Natl Acad Sci USA 1971, 68, 2374-2376, doi:10.1073/PNAS.68.10.2374.
[4] Khoubnasabjafari, M.; Mogaddam, M. R. A.; Rahimpour, E.; Soleymani, J.; Saei, A. A.; Jouyban, A. Breathomics: Review of Sample Collection and Analysis, Data Modeling and Clinical Applications. Crit Rev Anal Chem 2022, 52, 1461-1487, doi:10.1080/10408347.2021.1889961.
[5] Phillips, M.; Herrera, J.; Krishnan, S.; Zain, M.; Greenberg, J.; Cataneo, R. N. Variation in Volatile Organic Compounds in the Breath of Normal Humans. J Chromatogr B Biomed Sci Appl 1999, 729, 75-88, doi:10.1016/S0378-4347(99)00127-9.
[6] Thaler, E. R.; Hanson, C. W. Medical Applications of Electronic Nose Technology. Expert Rev Med Devices 2005, 2, 559-566, doi:10.1586/17434440.2.5.559.
[7] Amann, A.; Corradi, M.; Mazzone, P.; Mutti, A. Lung Cancer Biomarkers in Exhaled Breath. Expert Rev Mol Diagn 2011, 11, 207-217, doi:10.1586/ERM.10.112.
[8] Risby, T.; Tittel, F. Current Status of Midinfrared Quantum and Interband Cascade Lasers for Clinical Breath Analysis. Optical Engineering 2010, 49, 111123, doi:10.1117/1.3498768.
[9] Miekisch, W.; Schubert, J. K.; Noeldge-Schomburg, G. F. E. Diagnostic Potential of Breath Analysis—Focus on Volatile Organic Compounds. Clinica Chimica Acta 2004, 347, 25-39, doi:10.1016/j.cccn.2004.04.023.
[10] Lara-Ibeas, I.; Rodríguez Cuevas, A.; Le Calvé, S. Recent Developments and Trends in Miniaturized Gas Preconcentrators for Portable Gas Chromatography Systems: A Review. Sens Actuators B Chem 2021, 346, 130449, doi:10.1016/J.SNB.2021.130449.
[11] Lecharlier, A.; Carrier, H.; Le Hecho, I. Characterization of Biogas and Biomethane Trace Compounds: A Critical Review of Advances in in Situ Sampling and Preconcentration Techniques. Anal Chim Acta 2022, 1229, 340174, doi:10.1016/J.ACA.2022.340174.
[12] Wang, L.; Song, J. Research Progress of Aniline Vapor Sensor: A Review. Sens Actuators A Phys 2023, 362, 114676, doi:10.1016/J.SNA.2023.114676.
[13] Li, Y.; Hoi, K. I.; Mok, K. M.; Yuen, K. V. Air Quality Monitoring and Advanced Bayesian Modeling; 2023;
[14] Streibel, T.; Zimmermann, R. Resonance-Enhanced Multiphoton Ionization Mass Spectrometry (REMPI-MS): Applications for Process Analysis. Annu Rev Anal Chem (Palo Alto Calif) 2014, 7, 361-381, doi:10.1146/ANNUREV-ANCHEM-062012-092648.
[15] Grotemeyer, J.; Lindner, J.; Köster, C.; Schlag, E. W. Multiphoton Ionization Mass Spectrometry: Method and Applications. J Mol Struct 1990, 217, 51-68, doi:10.1016/0022-2860 (90) 80351-J.
[16] Dey, D.; Woodhouse, J. L.; Taylor, M. P.; Fielding, H. H.; Worth, G. A. On the Multiphoton Ionisation Photoelectron Spectra of Phenol. Physical Chemistry Chemical Physics 2024, doi:10.1039/D3CP05559K.
[17] Litani-Barzilai, I.; Bulatov, V.; Gridin, V. V.; Schechter, I. Detector Based on Time-Resolved Ion-Induced Voltage in Laser Multiphoton Ionization and Laser-Induced Fluorescence. Anal Chim Acta 2004, 501, 151-156, doi:10.1016/J.ACA.2003.09.025.
[18] Gridin, V. V.; Inoue, T.; Ogawa, T.; Schechter, I. ON-LINE SCREENING OF AIRBORNE PAH CONTAMINATION BY SIMULTANEOUS MULTIPHOTON IONIZATION AND LASER INDUCED FLUORESCENCE. Instrum Sci Technol 2000, 28, 131-141, doi:10.1081/CI-100100967.
[19] Litani-Barzilai, I.; Fisher, M.; Gridin, V. V.; Schechter, I. Fast Filter-Sampling and Analysis of PAH Aerosols by Laser Multiphoton Ionization. Anal Chim Acta 2001, 439, 1-8, doi:10.1016/S0003-2670(01)01026-1.
[20] Chen, Y.; Bulatov, V.; Vinerot, N.; Schechter, I. Letters to Analytical Chemistry Multiphoton Ionization Spectroscopy as a Diagnostic Technique of Surfaces Under Ambient Conditions. J.l.; Angel, S. M. Appl. Opt 1987, 59, 51, doi:10.1021/ac 100539x.
[21] Vinerot, N.; Chen, Y.; Bulatov, V.; Gridin, V. V.; Fun-Young, V.; Schechter, I. Spectral Characterization of Surfaces Using Laser Multi-Photon Ionization. Opt Mater (Amst) 2011, 34, 329-335, doi:10.1016/J.OPTMAT.2011.05.016.
[22] Tang, S.; Vinerot, N.; Fisher, D.; Bulatov, V.; Yavetz-Chen, Y.; Schechter, I. Detection and Mapping of Trace Explosives on Surfaces under Ambient Conditions Using Multiphoton Electron Extraction Spectroscopy (MEES). Talanta: The International Journal of Pure and Applied Analytical Chemistry 2016, 155, 235-244, doi:10.1016/J.TALANTA.2016.04.027.
[23] Kruve, A.; Bulatov, V.; Schechter, I. Quantitative and Sensitive Mapping of Imidacloprid on Plants Using Multiphoton Electron Extraction Spectroscopy. Chem Phys 2018, 514, 126-131, doi:10.1016/J.CHEMPHYS.2018.03.031.
[24] Tang, S.; Bulatov, V.; Schechter, I.; Dolgin, B. Parchment Characterization by Multiphoton Electron Extraction Spectrometry. Archaeometry 2022, 64, 468-482, doi:10.1111/ARCM.12723.
[25] Fisher, D.; Bulatov, V.; Schechter, I. Authentication and Testing of Banknotes Using Laser Multiphoton Electron Extraction Spectroscopy (MEES). 2021, doi:10.3390/app112110465.
[26] Fisher, D.; Bulatov, V.; Schechter, I. Application of Multiphoton Electron Extraction Spectroscopy (MEES) to Printed Document Forensics. Talanta Open 2022, 5, doi:10.1016/J.TALO.2022.100094.
[27] Tang, S.; Vinerot, N.; Bulatov, V.; Yavetz-Chen, Y.; Schechter, I. Multiphoton Electron Extraction Spectroscopy and Its Comparison with Other Spectroscopies for Direct Detection of Solids under Ambient Conditions. Anal Bioanal Chem 2016, 408, 8037-8051, doi:10.1007/S00216-016-9904-2.
[28] Tumbiolo, S.; Vincent, L.; Gal, J. F.; Maria, P. C. Thermogravimetric Calibration of Permeation Tubes Used for the Preparation of Gas Standards for Air Pollution Analysis. Analyst 2005, 130, 1369-1374, doi:10.1039/B508536E.
[29] Sartore, L.; Polvara, E.; Invernizzi, M.; Sironi, S. Determination of Air Pollutants: Application of a Low-Cost Method for Preparation of VOC Mixtures at Known Concentration. Sustainability (Switzerland) 2022, 14, 9149, doi:10.3390/SU14159149/S1.

What is claimed is:

1. A system for analyzing a gas, the system comprising:
a pair of electrodes arranged to generate an electric field therebetween;
a light source system configured to illuminate the gas between said electrodes by a light beam so as to release electrons from the gas by a multiphoton ionization process; and
a processor configured to receive from said electrodes an electrical signal generated by electrons accelerating within said electric field, to construct a multiphoton ionization spectrum based on said electrical signal, and to analyze the gas based on said spectrum.

2. The system according to claim 1, comprising a gas flow chamber, formed with a gas inlet and a gas outlet, wherein said electrodes are positioned in said gas flow chamber.

3. The system according to claim 1, wherein said light source system is configured to vary a wavelength of said light beam.

4. The system according to claim 2, wherein a pressure condition in an interior of said chamber is generally equal to a pressure condition outside said chamber.

5. The system according to claim 2, comprising a pump system configured to vary a pressure in said chamber.

6. The system according to claim 3, comprising a pump system configured to vary a pressure in said chamber, wherein said processor is configured to receive said electrical signal at different pressure conditions, for each of a plurality of different central wavelengths of said light beam, wherein for each of said central wavelengths, said analysis is based on changes of said signal in response to said pressure variation

7. The system according to claim 3, wherein said light source system comprises a plurality of light sources, each configured to generate light at a controllable wavelength.

8. The system according to claim 7, wherein said light source system is configured to illuminate said gas simultaneously by at least two light beams, each generated by a different light source and having a different wavelength.

9. The system according to claim 7, wherein said light source system is configured to generate at least two pulsed light beams, respectively from at least two of said light sources, wherein each pulsed light beam has a different wavelength and wherein pulses of said pulsed light beam are temporally interlaced with each other.

10. The system according to claim 2, wherein said gas inlet and gas outlet are opened at all times.

11. The system according to claim 2, wherein said gas flow chamber is telescopic and having a variable length.

12. The system according to claim 1, wherein a distance between said electrodes is controllable.

13. The system according to claim 1, wherein a location of the multiphoton ionization process between the electrode is controllable.

14. The system according to claim 1, wherein said processor is configured to construct a multiphoton ionization waveform, and to analyze the gas based on both said spectrum and said waveform.

15. The system according to claim 14, wherein said processor is configured to generate ion mobility data based on said waveform, and to analyze the gas based on both said spectrum and said ion mobility data.

16. The system according to claim 3, wherein said processor is synchronized with said light source system and is configured analyze said signal in bins, each bin corresponding to a different central wavelength of said light beam.

17. The system according to claim 16, wherein said processor is configured to apply time-integration separately to each bin, to provide a photocharge associated with said bin.

18. The system according to claim 2, wherein said gas flow chamber is a faraday cage chamber.

19. The system according to claim 1, comprising a lens positioned to focus said light beam, wherein a focal length of said lens is at least 10% of a length of said electrode along a propagation direction of said light beam.

20. The system according to claim 3, wherein said wavelength variation is at a sub-nanometer spectral resolution.
